(19) United States
(12) Patent Application Publication
(10) Pub. No.: US 2009/0177042 A1
(43) Pub. Date: Jul. 9, 2009

(54) Color image acquisition with scanning laser beam devices

(75) Inventor: Richard S. Johnston, Sammamush, WA (US)

Correspondence Address: BLAKELY SOKOLOFF TAYLOR & ZAFMAN LLP, 1279 OAKMEAD PARKWAY, SUNNYVALE, CA, 94085-4040, US

(73) Assignee: University of Washington

(21) Appl. No.: 12/008,324

(22) Filed: Jan. 9, 2008

(51) Int. Cl. A61B 1/00; G01N 21/27

(52) U.S. Cl. 600/178; 356/406

(57) ABSTRACT

An apparatus may include an interface to allow a scanning beam device to be coupled. The apparatus may include at least four lasers optically coupled with the interface. Each of the at least four lasers may provide a different wavelength visible spectrum light to the scanning beam device through the interface. An actuator driver of the apparatus may be electrically coupled with the interface. The actuator driver may provide actuator drive signals to the scanning beam device through the interface. The actuator drive signals may be operable to cause the scanning beam device to scan a beam of the different wavelength lights over a surface. An image generation unit of the apparatus may generate an image of the surface. The image may be generated based, at least in part, on light from the beam that has been reflected from the surface and detected by at least one photodetector.

### BACKGROUND

1. Field

Embodiments of the invention relate to image acquisition. In particular, embodiments of the invention relate to improved color image acquisition with scanning laser beam devices.

2. Background Information

Various scanning beam devices are known in the arts and described in the literature. One type of scanning beam device is a scanning beam image acquisition device, which may be used to acquire an image of a surface.

In acquiring the image, the device may scan a beam of light over the surface. Light from the beam may be backscattered or otherwise reflected from the surface. The reflected light may be collected and detected at different points in time throughout the scan while the beam is scanned over the surface. An image of the surface may be generated based, at least in part, on light detected at the different points in time.

The scanning beam image acquisition device may either collect monochromatic or color images. In the case of color images, the device typically includes a red light source, a blue light source, and a green light source (collectively an “RGB light source”). For example, the red light source may have a wavelength of about 635 nanometers (nm), the green light source may have a wavelength of about 532 nm, and the blue light source may have a wavelength of about 443 nm. The light sources are generally narrow-bandwidth laser light sources, which typically have a bandwidth of less than 5 nm.

However, the inventors recognize that there are limitations with using an RGB light source.

### BRIEF DESCRIPTION OF THE SEVERAL VIEWS OF THE DRAWINGS

The invention may best be understood by referring to the following description and accompanying drawings that are used to illustrate embodiments of the invention. In the drawings:

FIG. 1 is a block flow diagram of a method that may be performed by a scanning-beam color-image acquisition system, according to embodiments of the invention.

FIG. 2 is a block diagram of an example scanning-beam color-image acquisition system, according to embodiments of the invention.

FIG. 3 is a block flow diagram of a method that may be performed by a base station of a scanning-beam color-image acquisition system, according to embodiments of the invention.

FIG. 4 is a block diagram showing an example configuration of optical components of a base station, according to one or more embodiments of the invention.

FIG. 5 is a cross-sectional side view of a particular example of a suitable scanning fiber device, according to one or more embodiments of the invention.

### DETAILED DESCRIPTION

One limitation associated with using an RGB light source is that illuminating the surface with only narrow-bandwidth red, green, and blue light may result in certain colors being improperly acquired and represented in the image. For example, if a portion of the surface is yellow and only reflects light between the wavelengths of 550 to 600 nm, then the red, green, and blue lights from the RGB light source may each be absorbed instead of being reflected. As a result, the yellow portion of the surface would tend to appear black in the acquired image instead of yellow. In other words, the narrow bandwidth RGB light source may tend to limit the color fidelity of the acquired image relative to the surface, at least for certain surfaces having relatively narrow spectral response bands.

In the following description, numerous specific details are set forth. However, it is understood that embodiments of the invention may be practiced without these specific details. In other instances, well-known circuits, structures and techniques have not been shown in detail in order not to obscure the understanding of this description.

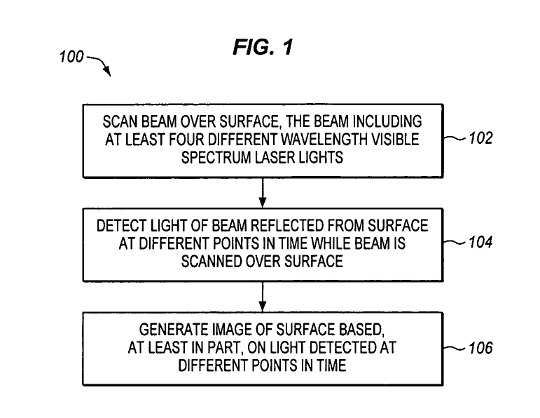

FIG. 1 is a block flow diagram of a method 100 that may be performed by a scanning-beam color-image acquisition system, according to embodiments of the invention.

At block 102, a beam may be scanned over a surface. Significantly, the beam may include at least four different wavelength visible spectrum laser lights. Recall that a beam from a conventional RGB light source only includes three colors or wavelengths, namely red, green, and blue.

The different laser lights may be separate, discrete, and/or non-continuous. In other words, they do not form a continuous band of light as there are gaps between them. The visible spectrum laser lights may have a bandwidth ranging from about 400 to 800 nm. In one or more embodiments of the invention, the bandwidths may be substantially evenly distributed, or at least somewhat spaced apart, over the visible spectrum range.

At block 104, light of the beam that has been reflected from the surface is detected. The light may be detected at different points in time while the beam is scanned over the surface.

At block 106, an image of the surface is generated. The image may be generated based, at least in part, on the light detected at the different points in time.

Advantageously, using the at least four different wavelength visible spectrum laser lights (instead of just three) may help to improve the color fidelity of the acquired image relative to the surface, at least for certain surfaces having relatively narrow spectral response bands. In other words, the acquired color image may more accurately represent the true colors in the surface. For example, including yellow laser light in the at least four laser lights may help to allow a surface that only reflects light between 550 to 600 nm appear yellow instead of the black color it would tend to appear if only the RGB light sources previously described in the background section were used.

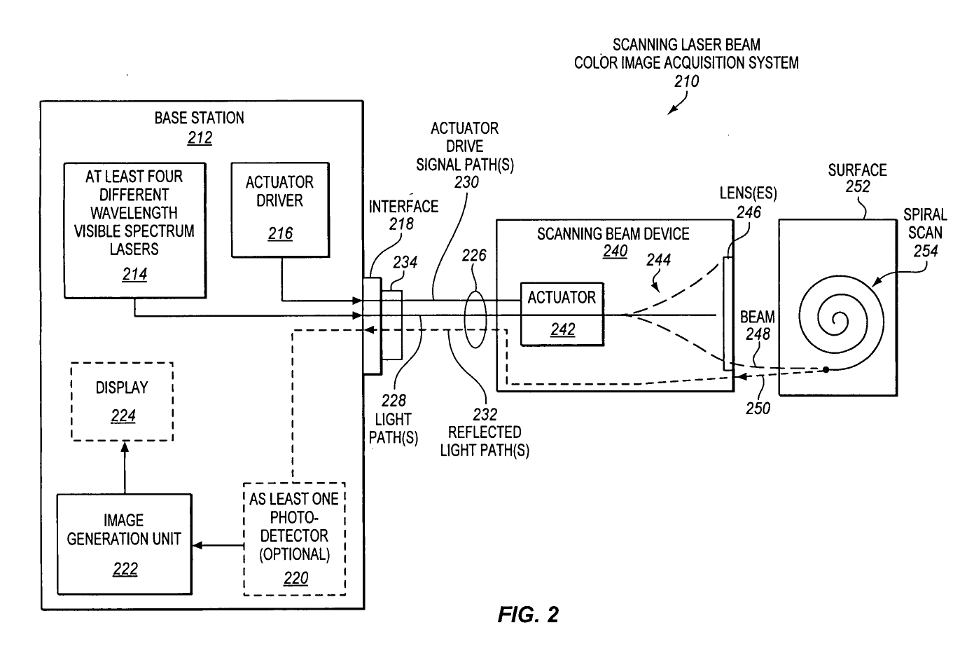

FIG. 2 is a block diagram of an example scanning laser beam color image acquisition system 210, according to embodiments of the invention. The system has a two-part form factor, although such a two-part form factor is not required. The two-part form factor includes a base station 212 and a scanning beam device 240.

The base station includes an interface 218. The interface may allow the scanning beam device 240 to be coupled.

The base station also includes at least four different wavelength visible spectrum lasers 214. The at least four lasers are optically coupled with the interface. Each of the at least four lasers are operable to provide a different wavelength visible spectrum laser light to the scanning beam device through the interface. Generally, each of the laser lights has a narrow bandwidth of less than 5 nm. As previously discussed, using at least four different wavelength visible spectrum laser lights (instead of just three) may help to improve color fidelity of an acquired image of a surface, at least for certain surfaces having relatively narrow spectral response bands.

Examples of suitable lasers include, but are not limited to, semiconductor lasers, solid state lasers (e.g., ruby lasers), gas lasers (e.g., helium-neon lasers and argon lasers), fiber hosted lasers, other lasers, and other narrow bandwidth sources of coherent light. Specific examples of suitable semiconductor lasers include, but are not limited to, laser diodes, double heterostructure lasers, quantum well lasers, separate confinement heterostructure lasers, distributed feedback lasers, vertical-cavity surface-emitting lasers (VCSELs), and combinations thereof.

In addition to the at least four different wavelength visible spectrum lasers, in one or more embodiments, the base station may also optionally include an infrared light source, an ultraviolet light source, a high intensity therapeutic laser light source (e.g., in the case of an endoscope), or a combination thereof. Depending on the implementation, the lasers or light sources may emit continuous streams of light, modulated light, or streams of light pulses.

Referring again to FIG. 2, the base station also includes an actuator driver 216. The actuator driver is electrically coupled with the interface. The actuator driver is operable to provide voltages or other electrical signals, which are referred to herein as actuator drive signals, to the scanning beam device through the interface. The actuator drive signals are operable to cause the scanning beam device to scan a beam 248 of the at least four different wavelength visible spectrum laser lights over a surface 252.

There are various different ways of implementing the actuator driver. The actuator driver may be implemented in hardware (for example a circuit), software (for example a routine or program), or a combination of hardware and software. As one example, in one or more embodiments of the invention, the actuator driver may include one or more lookup tables, or other data structures, stored in a memory or other storage location, which may provide previously stored actuator drive signal values. As another example, the actuator driver may include software executed by a computer or processor, or an application specific integrated circuit (ASIC), or other circuit, operable to generate the actuator drive signal values in real time. If desired, the actuator drive signal values may optionally be adjusted based on calibration, such as, for example, as described in U.S. Patent Application 20060072843, entitled “REMAPPING METHODS TO REDUCE DISTORTIONS IN IMAGES”, by Richard S. Johnston. The actuator drive signal values may be digital and may be provided to a digital-to-analog converter. One or more amplifiers may amplify the analog versions of the actuator drive signals. The amplified actuator drive signals may then be provided through the interface of the base station. These are just a few illustrative examples. Other actuator drivers will be apparent to those skilled in the art and having the benefit of the present disclosure.

Referring again to FIG. 2, the scanning beam device is shown to be electrically, optically, and physically coupled with the base station. In particular, the scanning beam device is coupled with the base station through one or more intervening cables 226. The one or more cables of the scanning beam device may have one or more connectors or other couplers to connect or otherwise couple with the interface 234.

As shown, the one or more cables may include at least one light path 228 to receive the at least four different wavelength visible spectrum laser lights from the at least four lasers, and convey the at least four different wavelength visible spectrum laser lights to the scanning beam device. The one or more cables may also include one or more drive signal paths 230 to receive the actuator drive signals from the actuator driver, and convey the actuator drive signals to the scanning beam device.

The scanning beam device includes an actuator 242 and a scanning optical element 244. The scanning optical element is optically coupled to receive the at least four different wavelength visible spectrum laser lights from the base station through the cable. The actuator is electrically coupled to receive the actuator drive signals from the base station through the cable.

In operation, the actuator may vibrate, or otherwise actuate or move the scanning optical element based on, and responsive to, the received actuator drive signals. The actuated scanning optical element may scan a beam of the at least four different wavelength visible spectrum laser lights through the one or more optional lenses 246 to scan a focused beam 248 over the surface 252. In embodiments of the invention, the actuator drive signals may be operable to cause the actuator to actuate the scanning optical element according to a two-dimensional scan. Examples of suitable two-dimensional scans include, but are not limited to, spiral scans, propeller scans, Lissajous scans, circular scans, oval scans, raster scans, and the like. In the illustration, a spiral scan 254 is shown, and a dot shows a position of the focused beam or illumination spot at a particular point in time during the scan.

One example of a suitable scanning optical element 244 is a single cantilevered free-end portion of an optical fiber. As shown, the single cantilevered free end portion is flexible and may be deflected during the scan. One example of a suitable actuator for the cantilevered free-end portion of the optical fiber is a piezoelectric tube, or other actuator tube, through which the optical fiber is inserted. The shape of the piezoelectric tube may be changed by the application of the electrical actuator drive signals to vibrate or move the flexible free end portion of the optical fiber according to the scan. Light may be emitted from a distal end or tip of the free end portion of the optical fiber while it is moved according to the scan.

Other scanning beam devices besides scanning fiber devices are also suitable. For example, the scanning beam device may include a mirror or other reflective device representing a scanning optical element, and a Micro-Electro-Mechanical System (MEMS), piezoelectric actuator, or other actuator, to move the reflective device to scan the beam. Still other scanning beam devices may include galvanometers, multiple optical elements moved relative to each other, or the like.

Light that is backscattered or otherwise reflected from the surface may be collected and detected at different points in time during the scan and used to generate an image of the surface. In the illustration, reflected light 250 from the beam or illumination spot is collected by the scanning beam device.

Different ways of collecting the reflected light are possible. One option is illustrated in FIG. 2. As shown, in one or more embodiments, the scanning beam device may optionally include one or more reflected light paths 232 to collect and convey the reflected light from a distal tip of the scanning beam device back to at least one photodetector. Examples of suitable types of photodetectors include, but are not limited to, photomultiplier tubes, photodiodes, phototransistors, other photodetectors known in the arts, and combinations thereof. As shown, the at least one photodetector 220 is optionally included in the base station in an optical path of reflected light returned from the scanning beam device through the interface.

As another option, in one or more embodiments, the scanning beam device may instead include the at least one photodetector proximate a distal tip thereof to detect the reflected light. The distal tip is closest to the surface. In this way there is no need for the reflected light paths 232, or for the base station to include the at least one photodetector 220. Rather, electrical signals representing the reflected light detected by these photodetectors of the scanning beam device may be conveyed back to the base station through the interface.

With reference again to FIG. 2, the base station also includes an image generation unit 222. The image generation unit is operable to generate an image of the surface. The image may be generated based, at least in part, on light from the beam that has been reflected from the surface and detected by at least one photodetector. For example, the image generation unit may generate the image by representing different pixels or other positions in the image with the amounts of light detected at different corresponding points in time during the scan. As shown in the illustration, the image generation unit may be electrically coupled with an output of the at least one optional photodetector 220 to receive electrical signals representing the detected light. As further shown, the base station may optionally include a display 224 to display the images. Alternatively, the display may be externally to the base station and capable of being coupled with the base station.

The described scanning-beam color-image acquisition system may take various forms and/or be used for different purposes. For example, in various illustrative embodiments of the invention, the system may take the form of a scanning beam or scanning fiber endoscope, boroscope, microscope, other type of scope, or other scanning beam or scanning fiber image acquisition system known in the art. In one particular example, the system may take the form of a scanning fiber endoscope system. As is known, an endoscope represents a device to be inserted into a patient to acquire images within a body cavity, lumen, or otherwise acquire images within the patient. For example, the scanning beam endoscope may be inserted into a patient, navigated through the patient to a surface of interest, and used to acquire an image of the surface. The image may be analyzed for medical or diagnostic purposes. Examples of suitable types of endoscopes include, but are not limited to, bronchoscopes, colonoscopes, gastroscopes, duodenoscopes, sigmoidoscopes, thorascopes, ureteroscopes, sinuscopes, boroscopes, and thorascopes, to name a few examples.

A simplified system has been shown and described in order to avoid obscuring the description. Other representative components that may be included in the base station include, but are not limited to, a power source, a user interface, and a memory. Furthermore, the base station may include supporting components like clocks, amplifiers, digital-to-analog converters, analog-to-digital converters, and the like.

To facilitate description, a system including the scanning beam device coupled with the base station has been used. However, the base station may be manufactured and/or sold separately from the scanning beam device. Accordingly, it is to be understood that embodiments of the invention pertain to a base station that may be, but does not need to be, coupled with a scanning beam device. Additionally, it is to be understood that the base stations described herein may be used with scanning beam devices other than those shown and described herein.

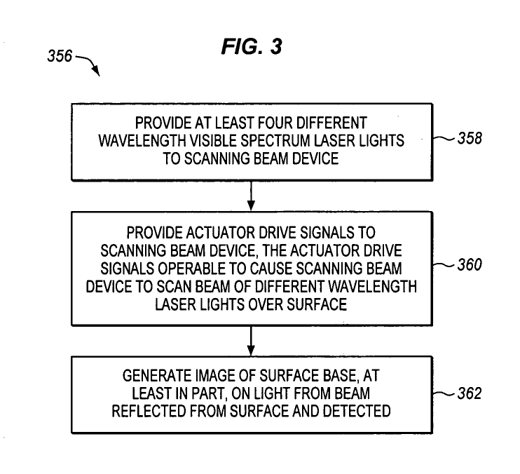

FIG. 3 is a block flow diagram of a method 356 that may be performed by a base station of a scanning-beam color-image acquisition system, according to embodiments of the invention.

At block 358, at least four different wavelength visible spectrum laser lights may be provided to a scanning beam device. As previously discussed, a conventional RGB light source only provides three colors, namely red, green, and blue.

At block 360, actuator drive signals may be provided to the scanning beam device. The actuator drive signals may be operable to cause the scanning beam device to scan a beam of the different wavelength visible spectrum laser lights over a surface.

At block 362, an image of the surface may be generated. The image may be generated based, at least in part, on light from the beam that has been reflected from the surface and detected.

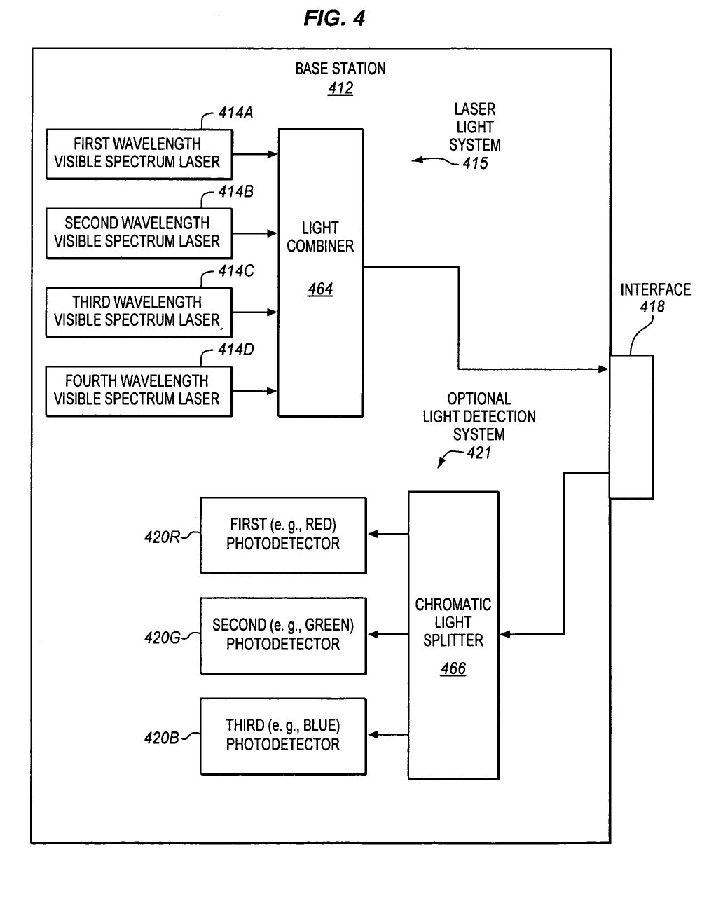

FIG. 4 is a block diagram showing an example configuration of optical components of a base station 412, according to one or more embodiments of the invention. Although not shown, it is to be understood that the base station may also include an actuator driver and other components and attributes as previously described.

The base station includes a laser light system 415 to provide at least four different wavelength visible spectrum laser lights. The laser light system includes a first wavelength visible spectrum laser 414A, a second wavelength visible spectrum laser 414B, a third wavelength visible spectrum laser 414C, and a fourth wavelength visible spectrum laser 414D. Examples of suitable lasers include, but are not limited to, the following:
(1) 440 nm Model NDHB510APAE1 laser diode from Nichia Corporation of Tokyo, Japan
(2) 470 nm Model NDHA210APAE1 laser diode from Nichia Corporation
(3) 532 nm Model BWN-532-20-SMF laser from B&W Tek Inc. of Newark, Deleware
(4) 543 nm Model 25LGR393 He—Ne laser from Melles Griot of Carlsbad, Calif.
(5) 568 nm Model 35KAP431 argon ion laser from Melles Griot
(6) 594 nm Model 25LYR173 He—Ne laser from Melles Griot
(7) 612 nm Model 25LOR151 He—Ne laser from Melles Griot
(8) 635 nm Model LPS-635 laser diode system from Thorlabs, Inc. of Newton, N.J.
(9) 660 nm Model LPS-660 laser diode system from Thorlabs, Inc.
(10) 675 nm Model LPS-675 laser diode system from Thorlabs, Inc.
(11) 685 nm Model FLD6A2TK laser diode from Thorlabs, Inc.

There are certain advantages to using red, green, and blue lasers. Accordingly, in one or more embodiments of the invention, the at least four different wavelength visible spectrum lasers include: (a) a first red laser, a first green laser, and a first blue laser; and (b) at least one laser selected from: (1) a second red laser having a different wavelength than the first red laser; (2) a second green laser having a different wavelength than the first green laser; (3) a second blue laser having a different wavelength than the first blue laser; and (4) a laser that is not one of a red laser, a green laser, and a blue laser. However, the use of red, green, and blue lasers is not required for other embodiments.

In one or more embodiments of the invention, the at least four lasers may include a laser having a wavelength that is reflected by a given predetermined surface of interest having a narrow spectral response band. Advantageously, this may help to ensure that the surface is properly represented in the image if light from the other of the at least four lasers are not reflected by this surface. For example, a laser with a specific wavelength may be included specifically to view a biological material that is otherwise not adequately viewed with an RGB light source.

In one or more embodiments, more than four lasers may optionally be included to each provide a different wavelength visible spectrum laser light. For example, five, six, ten, or more lasers may optionally be included.

The laser light system also includes a light combiner 464. Each of the lasers is optically coupled with the light combiner, for example, through a separate singlemode optical fiber. The light combiner is optically coupled between the at least four lasers and an interface 418 of the base station. The light combiner may combine the at least four different wavelength visible spectrum laser lights into a combined laser light and provide the combined light to the interface. In one aspect, the light combiner may be designed analogously to the 635/532/440 RGB Combiner, which is available from SIFAM Fibre Optics Ltd., of Devon, United Kingdom but to accommodate at least one other wavelength of laser light. This may be readily done by those skilled in the art and having the benefit of the present disclosure. If desired, SIFAM may be contracted for a custom light combiner. As another option, the light combiner may be designed as the reverse of a light splitter.

To better illustrate certain concepts, the illustrated base station also includes an optional light detection system 421. However, as previously discussed, the light detection system may optionally be included in the scanning beam device, or otherwise not be included in the base station.

The illustrated light detection system includes a wide bandwidth chromatic light splitter 466, which is optional as discussed further below. By way of example, the light splitter may include a conventional assembly of focusing optics and dichroic beam splitters. Examples of suitable chromatic light splitters are Z440RDC and Z532RDC dichroic beam splitters, available from Chroma Technology Corporation, of Rockingham, Vt.

The chromatic light splitter is optically coupled with the interface. The light splitter may receive reflected light returned through the interface from a scanning beam device that is coupled with the interface. The light splitter may split the reflected light into a plurality of different colored portions or wavelengths. As shown in the illustrated embodiment, the plurality may be three. In one or more embodiments, the three different portions may be red, green, and blue portions or wavelengths. As used herein “red”, “green”, and “blue” do not imply any strict bandwidth, but rather are intended to cover light which is relatively “redish”, “greenish”, or “blueish”.

Notice that the chromatic light splitter may potentially split the reflected light into a lesser number of differently colored portions than a number of the at least four different wavelength visible spectrum lasers or laser lights used for illumination. In some cases, the chromatic light splitter may potentially split the reflected light into just three differently colored portions (for example red, green, and blue portions or wavelengths), regardless of whether a number of the at least four lasers or laser lights is at least four, at least six, at least ten, or more than ten.

The base station also includes a plurality of photodetectors. In particular, the base station includes a first (e.g., red) photodetector 420R, a second (e.g., green) photodetector 420G, and a third (e.g., blue) photodetector 420B. Notice that there are fewer photodetectors (three) than lasers (at least four). Each of the first, second, and third photodetectors is optically coupled with an output of the chromatic light splitter to receive a corresponding split light. An example of a suitable photodetector is H7826 photomultiplier tube module, which is available from Hamamatsu Photonics K.K., of Japan.

It is not required that there be only three photodetectors. As yet another option, the light detection system may optionally split the light into a fourth colored portion or wavelength and include a fourth photodetector to detect laser light having a wavelength corresponding to a fourth of the at least four lasers.

Alternate light detection systems are also suitable. For example, in one or more embodiments, first (e.g., red), second (e.g., green), and third (e.g., blue) wide bandwidth optical filters (not shown) may be optically coupled between the respective first, second, and third photodetectors and the interface. Each filter may filter out light that is to be detected by a non-corresponding photodetector and accordingly not to be detected by the corresponding photodetector. For example, a red filter corresponding to a red photodetector may filter out light that is to be detected by the respective blue and green photodetectors.

In one aspect, the filters may receive light split by a conventional light or beam splitter. However, using such a beam splitter may reduce the amount of light detected. As another option, rather than splitting the light, a first set of one or more optical fibers may be used to convey reflected light from the interface to the first (e.g., red) filter, a second set may be used to convey reflected light from the interface to the second (e.g., green) filter, and a third set may be used to convey reflected light from the interface to the third (e.g., blue) filter. However, this approach may also tend to reduce the amount of light detected.

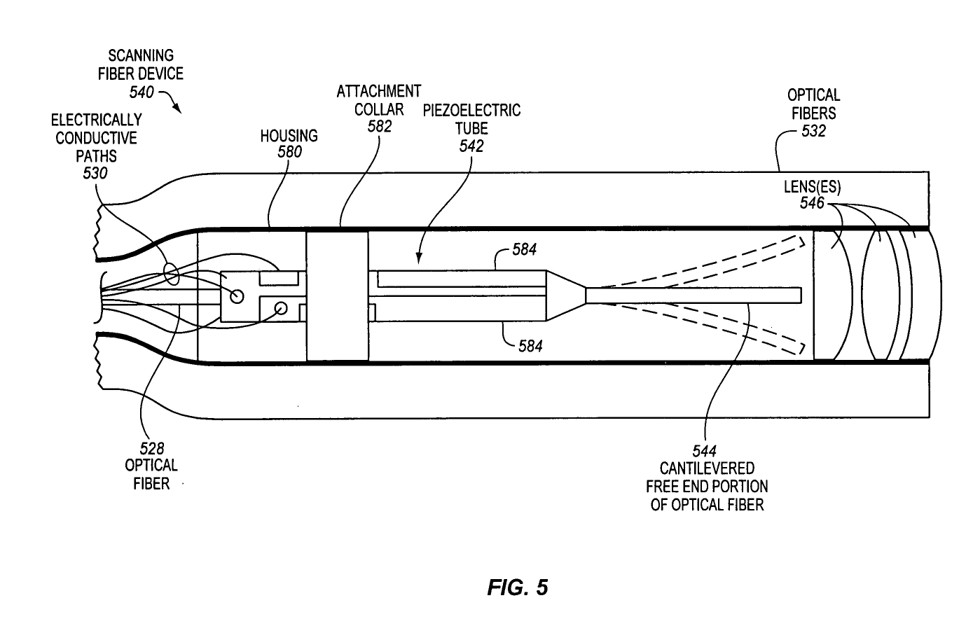

FIG. 5 is a cross-sectional side view of a particular example of a suitable scanning fiber device 540, according to one or more embodiments of the invention. The design of this device is well suited for use as an endoscope or other relatively small device, although in other implementations the design and/or operation may vary considerably.

The scanning fiber device includes a housing 580. In one or more embodiments, the housing may be relatively small and hermetically sealed. For example, the housing may be generally tubular, have a diameter that is about 5 millimeters (mm) or less, and have a length that is about 20 mm or less. In one or more embodiments, the diameter may be about 1.5 mm or less, and the length may be about 12 mm or less. The housing typically includes one or more lenses 546. Examples of suitable lenses include those manufactured by Hoya Corporation of Tokyo Japan, although other lenses may optionally be used.

As shown, one or more optical fibers 532 may optionally be included around the outside of the housing to collect and convey reflected light back to one or more photodetectors, for example, located in a base station. Alternatively, one or more photodetectors may be included at or near a distal tip of the scanning fiber device.

A piezoelectric tube 542, representing one possible type of actuator, is included in the housing. By way of example, the piezoelectric tube may include a PZT 5A material, although this is not required. Suitable piezoelectric tubes are commercially available from: Morgan Technical Ceramics Sales, of Fairfield, N.J.; Sensor Technology Ltd., of Collingwood, Ontario, Canada; and PI (Physik Instrumente) L.P., of Auburn, Mass. The piezoelectric tube may be inserted through a tightly fitting generally cylindrical opening of an attachment collar 582 that is used to attach the piezoelectric tube to the housing.

A portion of an optical fiber 528 is inserted through a generally cylindrical opening in the piezoelectric tube. A cantilevered free end portion 544 of the optical fiber extends beyond an end of the piezoelectric tube within the housing, and may be attached to the end of the piezoelectric tube, for example, with an adhesive.

The piezoelectric tube has electrodes 584 thereon. Wires or other electrically conductive paths 530 are electrically coupled with the electrodes to convey actuator drive signals to the electrodes. As shown, in one example embodiment, the piezoelectric tube may have four, quadrant metal electrodes on an outer surface thereof. Four wires may respectively be electrically coupled with the four electrodes. An optional ground electrode may be included on an inside surface of the piezoelectric tube.

Responsive to the actuator drive signals, the electrodes may apply electric fields to the piezoelectric tube. The electric fields may cause the piezoelectric tube to actuate the optical fiber. While the free end portion of the optical fiber may be vibrated at various frequencies, in one or more embodiments, it may be vibrated at or proximate, for example within a Q-factor of, its resonant frequency, or harmonics of the resonant frequency. As is known, the Q-factor is the ratio of the height to the width of the resonant gain curve for the free end portion of the optical fiber. Due to the increased gain around the resonant frequency, vibrating the free end portion of the optical fiber at or around the resonant frequency may help to reduce the amount of energy, or magnitude of the actuator drive signal, needed to achieve a given displacement, or perform a given scan.

The four quadrant electrodes, or even only two electrodes, may be capable of moving the optical fiber in a two-dimensional scan. By way of example, to move the cantilevered optical fiber in a spiral scan, equal frequency, increasing amplitude, out-of-phase sinusoidal, actuator drive signals may be applied to the electrodes. Further background information on suitable forms of scanning, if desired, is available in U.S. Patent Application 20060138238, entitled “METHODS OF DRIVING A SCANNING BEAM DEVICE TO ACHIEVE HIGH FRAME RATES”, by Richard S. Johnston et al. In the description and claims, the terms “coupled” and “connected,” along with their derivatives, are used. It should be understood that these terms are not intended as synonyms for each other. Rather, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but may still co-operate or interact with each other, for example, through one or more intervening components. As one example, the at least four lasers may be optically coupled with the interface through intervening optical fibers or other optical paths. As another example, the image generation unit may be coupled with the interface through at least one intervening component (e.g., at least one photodetector).

In the description and claims, the term “scanning” as in “scanning beam device”, and the like, does not necessarily imply that the device is in use, or presently in the process of “scanning”. Rather, the term “scanning” merely implies capability of scanning.

In the description above, for the purposes of explanation, numerous specific details have been set forth in order to provide a thorough understanding of the embodiments of the invention. It will be apparent however, to one skilled in the art, that embodiments may be practiced without these specific details. The particular embodiments are not provided to limit the invention but rather to illustrate it. The scope of the invention is not to be determined by the specific examples provided above but only by the claims below. All equivalent relationships to those illustrated in the drawings and described in the specification are encompassed within embodiments of the invention.

In other instances, well-known circuits, structures, devices, and operations have been shown in block diagram form or without detail in order to avoid obscuring the understanding of the description. Where considered appropriate, reference numerals or terminal portions of reference numerals have been repeated among the figures to indicate corresponding or analogous elements, which may optionally have similar characteristics.

Various operations and methods have been described. The methods have been described in basic form, but operations may optionally be added to the methods. In some cases, the operations of the methods may be changed, or operations may be removed from the methods, or the operations may be performed in different order. Certain operations may be performed by hardware components, or may be embodied in machine-executable instructions, that may be used to cause, or at least result in, a circuit, apparatus, or system programmed with the instructions performing the operations. Also the operations may optionally be performed by a combination of hardware and software.

For clarity, in the claims, any element that does not explicitly state “means for” performing a specified function, or “step for” performing a specified function, is not to be interpreted in the United States as a “means” or “step” clause as specified in 35 U.S.C. Section 112, Paragraph 6.

It should also be appreciated that reference throughout this specification to “one embodiment”, “an embodiment”, or “one or more embodiments”, for example, means that a particular feature may be included in the practice of the invention. Similarly, it should be appreciated that in the description various features are sometimes grouped together in a single embodiment, Figure, or description thereof for the purpose of streamlining the disclosure and aiding in the understanding of various inventive aspects. This method of disclosure, however, is not to be interpreted as reflecting an intention that the invention requires more features than are expressly recited in each claim. Rather, as the following claims reflect, inventive aspects may lie in less than all features of a single disclosed embodiment. Thus, the claims following the Detailed Description are hereby expressly incorporated into this Detailed Description, with each claim standing on its own as a separate embodiment of the invention.

What is claimed is:

1. An apparatus comprising:
at least four lasers, each of the at least four lasers to provide a different wavelength visible spectrum laser light;
a scanning beam device to scan a beam of the at least four different wavelength visible spectrum laser lights over a surface; and
an image generation unit to generate an image of the surface based at least in part on light from the beam that has been reflected from the surface and detected by at least one photodetector.

2. The apparatus of claim 1, wherein the at least four lasers comprise:
(a) a first red laser, a first green laser, and a first blue laser; and
(b) at least one laser selected from: (1) a second red laser, the second red laser having a different wavelength than the first red laser; (2) a second green laser, the second green laser having a different wavelength than the first green laser; (3) a second blue laser, the second blue laser having a different wavelength than the first blue laser; and (4) a laser that is not one of a red laser, a green laser, and a blue laser.

3. The apparatus of claim 1, further comprising the at least one photodetector, wherein the at least one photodetector includes a number of photodetectors that is less than a number of the at least four lasers.

4. The apparatus of claim 1, wherein the at least four lasers comprise a laser having a wavelength that is reflected by a predetermined surface of interest having a narrow spectral response band that is known in advance not to reflect any of the other lasers of the at least four lasers.

5. An apparatus comprising:
an interface to allow a scanning beam device to be coupled;
at least four lasers optically coupled with the interface, each of the at least four lasers to provide a different wavelength visible spectrum laser light to the scanning beam device through the interface;
an actuator driver electrically coupled with the interface, the actuator driver to provide actuator drive signals to the scanning beam device through the interface, the actuator drive signals operable to cause the scanning beam device to scan a beam of the different wavelength visible spectrum laser lights over a surface; and
an image generation unit to generate an image of the surface based at least in part on light from the beam that has been reflected from the surface and detected by at least one photodetector.

6. The apparatus of claim 5, wherein the at least four lasers comprise:
(a) a first red laser, a first green laser, and a first blue laser; and
(b) at least one laser selected from: (1) a second red laser, the second red laser having a different wavelength than the first red laser; (2) a second green laser, the second green laser having a different wavelength than the first green laser; (3) a second blue laser, the second blue laser having a different wavelength than the first blue laser; and (4) a laser that is not one of a red laser, a green laser, and a blue laser.

7. The apparatus of claim 5, wherein the at least four lasers comprise at least five lasers to each provide a different wavelength visible spectrum laser light.

8. The apparatus of claim 5, further comprising a light combiner coupled between the at least four lasers and the interface, the light combiner to combine the different wavelength laser lights into combined light and provide the combined light to the interface.

9. The apparatus of claim 5, further comprising the at least one photodetector, wherein the at least one photodetector is in an optical path of reflected light returned from the scanning beam device through the interface.

10. The apparatus of claim 9, wherein the at least one photodetector includes a number of photodetectors that is less than a number of the at least four lasers.

11. The apparatus of claim 10, wherein the number of photodetectors is three, and wherein the three photodetectors comprise a red photodetector, a green photodetector, and a blue photodetector.

12. The apparatus of claim 10, further comprising a light splitter optically coupled between the interface and the number of photodetectors, the light splitter to split the reflected light into different wavelength portions.

13. The apparatus of claim 5, wherein the at least four lasers comprise a laser having a wavelength that is reflected by a predetermined surface of interest having a narrow spectral response band that is known in advance not to reflect any of the other lasers of the at least four lasers.

14. The apparatus of claim 5, further comprising the scanning beam device coupled with the interface, wherein the scanning beam device comprises a scanning beam endoscope.

15. The apparatus of claim 5, further comprising the scanning beam device coupled with the interface, wherein the scanning beam device comprises a scanning fiber device having an actuator and a single cantilevered optical fiber, and wherein the actuator drive signals are operable to cause the actuator to vibrate the optical fiber within a Q-factor of its resonant frequency.

16. A method comprising:
providing at least four different wavelength visible spectrum laser lights to a scanning beam device;
providing actuator drive signals to the scanning beam device, the actuator drive signals operable to cause the scanning beam device to scan a beam of the different wavelength visible spectrum laser lights over a surface; and
generating an image of the surface based at least in part on light from the beam that has been reflected from the surface and detected.

17. The method of claim 16, wherein providing the at least four different wavelength visible spectrum laser lights comprises:
(a) providing a first red laser light, providing a first green laser light, and providing a first blue laser light; and
(b) providing at least one laser light selected from: (1) a second red laser light, the second red laser light having a different wavelength than the first red laser light; (2) a second green laser light, the second green laser light having a different wavelength than the first green laser light; (3) a second blue laser light, the second blue laser light having a different wavelength than the first blue laser light; and (4) a laser light that is not one of a red laser light, a green laser light, and a blue laser light.

18. The method of claim 16, further comprising detecting the reflected light with a number of photodetectors that is less than a number of the different wavelength laser lights.

19. A method comprising:
scanning a beam over a surface, the beam including at least four different wavelength visible spectrum laser lights;
detecting light of the beam that has been reflected from the surface at different points in time while the beam is scanned over the surface; and
generating an image of the surface based at least in part on the light detected at the different points in time.

20. The method of claim 19, wherein scanning comprises scanning a beam that comprises:
(a) a first red laser light, a first green laser light, and a first blue laser light; and
(b) at least one laser light selected from: (1) a second red laser light having a different wavelength than the first red laser light; (2) a second green laser light having a different wavelength than the first green laser light; (3) a second blue laser light having a different wavelength than the first blue laser light; and (4) a laser light that is not one of a red laser light, a green laser light, and a blue laser light.

21. The method of claim 19, wherein detecting comprises detecting the light with a number of photodetectors that is less than a number of the distinct, different wavelength visible spectrum laser lights.

22. The method of claim 19, wherein scanning the beam comprises scanning a laser light having a wavelength that is reflected by a predetermined surface of interest having a narrow spectral response band that is known in advance not to reflect any of the other lasers of the at least four lasers.

23. The method of claim 19, wherein scanning the beam comprises vibrating a single cantilevered optical fiber within a Q-factor of its resonant frequency.

24. The method of claim 19, further comprising inserting a scanning beam endoscope into a patient, and wherein scanning the beam over the surface comprises scanning the beam over a surface within the patient with the scanning beam endoscope.
